(19) United States
(12) Patent Application Publication
(10) Pub. No.: US 2013/0211221 A1
(43) Pub. Date: Aug. 15, 2013

(54) SYSTEM AND METHOD FOR IDENTIFYING AND COMMUNICATING WITH AN INTERVENTIONAL MEDICAL DEVICE

(71) Applicant: IMRICOR MEDICAL SYSTEMS, INC., (US)

(72) Inventors: Daniel N. Sunnarborg, Bloomington, MN (US); Peter S. Gabrail, Minneapolis, MN (US); Douglas A. Page, Apple Valley, MN (US); Steven R. Wedan, Savage, MN (US)

(73) Assignee: IMRICOR MEDICAL SYSTEMS, INC., Burnsville, MN (US)

(21) Appl. No.: 13/762,958

(22) Filed: Feb. 8, 2013

Related U.S. Application Data
(60) Provisional application No. 61/596,551, filed on Feb. 8, 2012.

(51) Int. Cl. A61B 5/00

(52) U.S. Cl. CPC A61B 5/742
USPC 600/373

(57) ABSTRACT

A system and method of identifying and communicating with an interventional medical device is provided. The system includes a novel catheter handle operably coupled to the shaft of a catheter at the distal end and an electrophysiology recording system at the proximal end. The catheter handle includes a visual identification system visible through a lens on the catheter handles and a microcontroller with a memory structure, the memory structure including catheter identification information. The visual identification system includes a light emitting diode, which is visible from the outside of the catheter handle through the lens. When a user of the EP recording system selects a desired color for the coupled catheter, the desired color is communicated to the microcontroller in the catheter handle and the microcontroller causes the visual identification to display the requested color so that the desired color is visible by the user.

### FIELD OF THE INVENTION

The invention relates to medical devices used in diagnostic and therapeutic procedures. In particular, the invention relates to a system and method for identifying and communicating with an interventional medical device while in use.

### BACKGROUND OF THE INVENTION

Modern interventional procedures involve the simultaneous use of multiple catheters such as one or more electrophysiology catheters, balloon catheters, delivery catheters, ablative catheters, diagnostic catheters, cardiac catheters, catheters to monitor pressures and other bodily functions and the like. Exemplary interventional procedures include, for example, cardiac electrophysiology procedures including diagnostic procedures for diagnosing arrhythmias and ablation procedures such as atrial fibrillation ablation, ventricular tachycardia ablation, atrial flutter ablation, Wolfe Parkinson White Syndrome ablation, AV node ablation, SVT ablations and the like. In these types of procedures, each different catheter may be used for a different purpose and/or placed in a different location or cavity within the body. Modern interventional tools require the user to configure the electrophysiology (EP) system such that the individual catheters in use may be easily identified on the EP system display, and the data provided by the EP system may be correlated to the functionality of the catheter. Additionally, the catheters require the user to manually change an identification mechanism, such as adding a label or a colored marker to the handle to distinguish one catheter from another. Information about the type of catheter is then manually entered into the EP recording system, or possibly using a technique including RFID. Different manufacturers use different techniques and methods for identifying both the physical catheter and differentiating the different catheters on the EP system display. This can result in confusion and an increase in procedure duration while the EP system is reconfigured and/or the particular catheter is replaced with the correct one.

An increase in procedure time directly correlates to an increased risk to the patient, while misidentification of a catheter may result in an improperly configured catheter; use of a catheter beyond it useful life or expiration data; providing inaccurate or misleading information to the physician; use of incorrect calibration data when configuring the catheter.

The available conventional art offers only partial solutions to the problem of identifying and configuring catheters and other tools utilized during an interventional procedure. While the use of RFID to communicate various information related to the function of a catheter has previously been disclosed, such use is not free from complications associated with RF transmit and receive antennas, such as interference and loss of signal. Additionally, conventional systems and method for implementing a data management system for catheters do not address the need for visually identifying catheters.

Therefore, what is needed is a system and method that would minimize the duration of a procedure and at the same time maximize the proper identification of each catheter in use and their respective associated functionality. What is also needed is a system and method that does not rely on RFID to communicate between the catheter and the EP system. Further, a solution for dynamic visual catheter identification is needed.

### BRIEF SUMMARY OF THE INVENTION

The system and method for identifying and communicating with an interventional medical device, such as a catheter, addresses the problems associated with the currently available conventional art.

The system and method in accordance with the invention reduces the potential for confusion or misidentification of a catheter by equipping the EP lab with the ability to identify catheters electronically.

The system and method in accordance with the invention further reduces the potential for confusion or misidentification of a catheter by equipping the EP lab with the ability to identify catheters simply and accurately by sight.

The system and method in accordance with the invention provides the ability to automatically configure catheters to be used during the interventional procedure and the EP recording system via an electronic control system and display a visual indicator identifying the catheter.

The system and method in accordance with the invention results in a faster work-flow in an environment where any decrease in procedure time is beneficial to the patient.

The system and method in accordance with the invention advantageously provides a solution for dynamic visual catheter identification.

In one aspect of the invention a system and method of managing catheter data is disclosed. The system and method includes coupling a catheter to an EP system. The catheter includes a microcontroller, which contains both a memory structure containing information about the catheter and a communications interface. The communications interface couples with a matched communications interface in the EP system. This permits the EP system to retrieve and update the memory structures in the catheter handle, retrieving information about the catheter, including, but not limited to, catheter type, serial number, expiration date, lot number, and physical characteristics

In another aspect of the invention the catheter handle includes a visual identification system. The visual identification system comprises a light emitting diode (LED) or electronic display. A microcontroller in the catheter handle controls the LED or electronic display and provides a visual, uniquely identifiable state, including, but not limited to colors, symbols, or textual information. When a catheter is coupled to an electrophysiology recording system, the electrophysiology recording system transmits an electronic message to the microcontroller requesting a desired visual state for the catheter. The microcontroller in turn causes the visual identification system to display the visual state to the user.

The system in accordance with one aspect of the invention broadly comprises an electrophysiology recording system operably coupled to a catheter having a catheter handle. The catheter handle includes a communication interface that facilitates communication between the catheter and an electrophysiology recording system via a bi-directional cable. The catheter handle includes an integrated circuit board including a microcontroller. The microcontroller includes a memory structure that contains catheter identification and other informational data about the catheter.

The system in accordance with another aspect of the invention includes a novel catheter handle operably coupled to a shaft of a catheter at the distal end and an electrophysiology recording system at the proximal end. The novel catheter handle includes a circuit board having a microcontroller with a memory structure, the memory structure including catheter identification information. The microcontroller controls an electronic identification mechanism comprising a light emitting diode (LED) positioned on the catheter handle. The LED is visible from the outside of the catheter handle through a transparent or semi-transparent lens. When a user of the EP recording system selects a desired color, the desired color is communicated to the microcontroller and the microcontroller causes the LED to display the requested color so that the desired color is visible by the user.

These and other features of the invention may be more clearly understood in view of the following detailed description of an embodiment of the invention.

### BRIEF DESCRIPTION OF THE DRAWINGS

FIG. 1A is a perspective view of the system in accordance with the invention.

FIG. 1B is an enlarged view of the circled area of FIG. 1 showing a perspective view of the catheter handle in accordance with the invention.

FIG. 2 is a cut away view of the catheter handle in accordance with the invention.

FIG. 3 is an exploded view of the catheter handle in accordance with the invention.

FIG. 4 is a flow chart depicting the method of identifying an interventional medical device in accordance with the invention.

### DETAILED DESCRIPTION OF THE INVENTION

A novel device and method of identifying catheters and configuring an interventional system that allows for both visually marking catheters and automatically configuring a EP recording system is disclosed.

The system in accordance with one aspect of the invention broadly comprises an electrophysiology recording system operably coupled to a catheter having a catheter handle. The catheter handle includes a communications interface that facilitates communication between the catheter and an electrophysiology recording system via a bi-directional cable. The catheter handle includes an integrated circuit board including a microcontroller. The microcontroller includes a memory structure that contains catheter identification and other informational data about the catheter.

The system in accordance with another aspect of the invention includes a novel catheter handle operably coupled to a shaft of a catheter at the distal end and an electrophysiology recording system at the proximal end. The novel catheter handle includes a circuit board having a microcontroller with a memory structure, the memory structure including catheter identification information. The microcontroller controls an electronic visual identification mechanism, which displays visual information that is visible from the outside of the catheter handle through a transparent or semi-transparent lens. The visual identification mechanism may comprise a light emitting diode. When a user of the electrophysiology recording system selects a desired color or other visual textual or graphic display, the desired color or other visual textual or graphic display information is communicated to the microcontroller in the catheter handle, which in turn causes the visual identification mechanism to display the requested visual state which will be visible by the user.

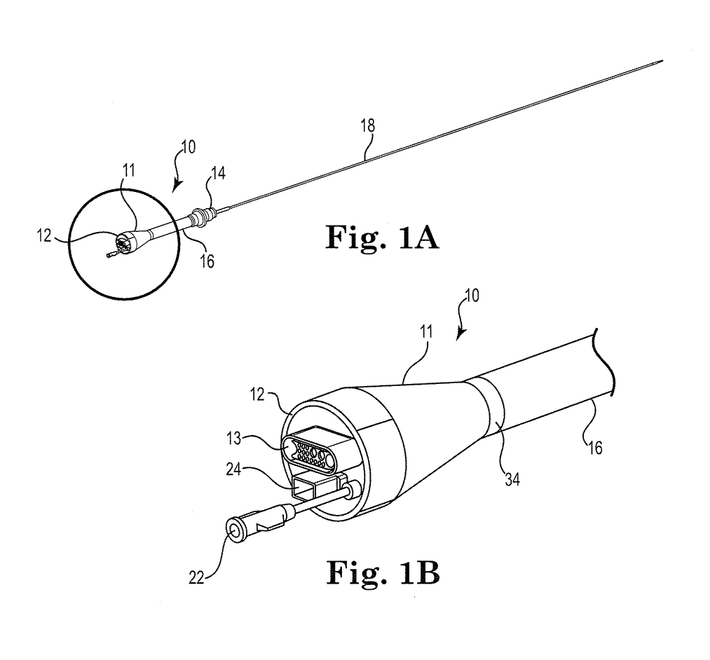

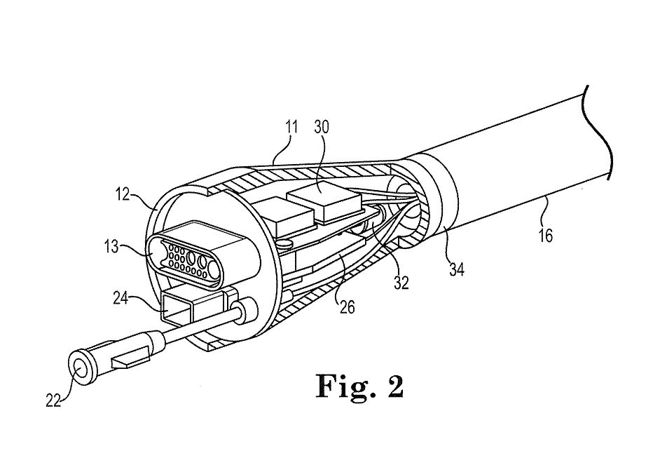

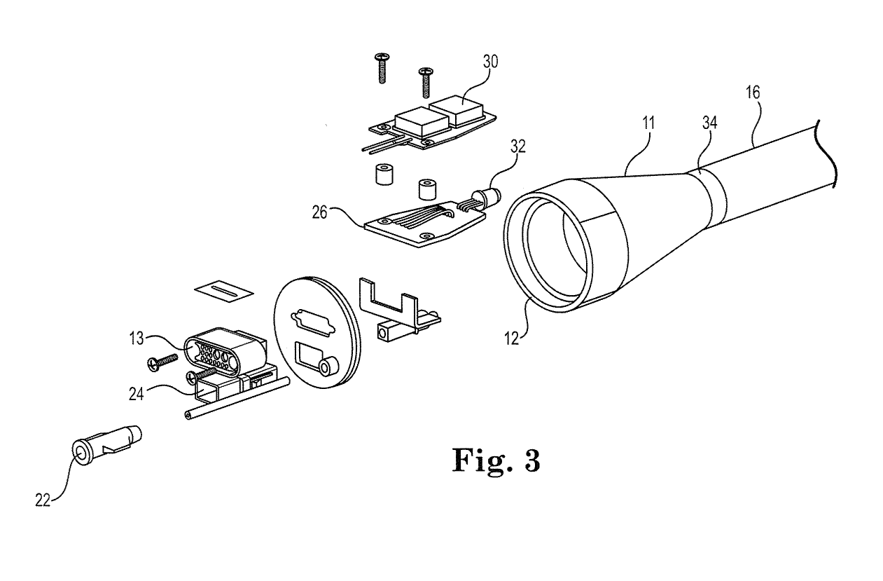

Referring now to FIGS. 1-3, the catheter handle 10 in accordance with the invention includes housing 11, a proximal end 12 and a distal end 14. The distal end 14 is operably coupled to a hand-gripping portion 16 of a catheter 18. The proximal end 12 of the catheter handle 10 comprises a communications interface 13 that allows the catheter handle 10 to be electrically coupled to an EP recording system (not shown) via a connection cable (not shown). Irrigation port 22 operably couples to a source of irrigation, such as a saline solution, at the distal end and the lumen of catheter 18 at the proximal end. Optical fiber connection 24 is used for measuring the temperature at the catheter tip.

The catheter handle 10 includes circuit board 26 within housing 11. The circuit board 26 includes a microcontroller/microprocessor (not shown) thereon. Microcontroller includes a memory structure for storing information about the subject catheter 18. A non-limiting list of such information includes model of the catheter, type, configuration, optional features, calibration data, calibration constants, serial number of the catheter, revision, build date, expiration date, date of last use, hours of use, permissible use duration, permissible number of uses, patient information, failure codes, single use enforcement, calibration settings, statistical information and other like information known to those of skill in the art. The memory may be volatile, non-volatile, removable or non-removable memory or may comprise any type of computer storage media which can be used to store the desired information about the catheter and which can be accessed by a computing device, such as an EP system. Those of skill in the art will appreciate that information relevant to the operation of the catheter 18 may be stored in non-volatile memory.

The microcontroller provides bidirectional communication with the EP recording system. Thus, the microcontroller and the peripheral EP recording system work as a transmitter/receiver pair or vice versa. Methods of communication between the microcontroller and the EP system may include both wired and wireless (e.g., RF, optical, or infrared) communications methods. In one aspect of the invention, the microcontroller provides bidirectional communication with the EP recording system through a cable. The EP recording system includes a control processor that is operable to retrieve data from the memory structure. The data retrieved by the EP recording system contains at least one characteristic relevant to the functionality of the catheter. In operable bi-directional communication, the EP system automatically configures itself based on the data retrieved from the memory structure and the data stored in the memory structure is updated by the EP recording system.

The catheter handle also includes handle card components and circuitry 30 and an electronic visual identification system 32. Circuitry 30 may contain, for example, transformers for de-coupling a tracking coil transmission line from an external coaxial transmission line. The microcontroller communicates with the multi-color LED by sending a signal to display a particular color. The microcontroller may control the red, green and blue levels of the LED and as a result, the LED displays the chosen color.

The electronic visual identification mechanism 32 comprises a multicolored light emitting diode (LED). The LED allows the system in accordance with the invention to assign a color, such as red, blue, green, etc., to the catheter handle that uniquely identifies it. Housing 11 includes light lens 34, which is transparent or semi-transparent. The color of the LED is thus capable of being viewed through the light lens 34 of the catheter handle 10 in proximity to the LED 32. Suitable materials for the light lens may include polycarbonate and other polymers known to those of skill in the art. The color of the LED may correlate to an identifiable feature on the EP recording system such as the color of the trace of electrogram produced by the catheter when the catheter is positioned within a patient. As those of skill in the art will appreciate, the LED may also be configured to communicate visual information regarding the catheter status to the surgeon using the catheter. For example, blinking of the LED may be a signal for communicating a failure code and the necessity to change out the catheter.

Alternatively, the electronic visual identification system 32 may include a configurable textual or graphic display on the catheter handle 10 itself; through the lens or on the catheter 18. In another aspect of the invention, the electronic identification system 32 may include both an LED visible through a transparent window and a configurable textual or graphic display. The configurable textual or graphic display is used for catheter identification and may be used to communicate information regarding the catheter status such as catheter failure, single use enforcement and the like.

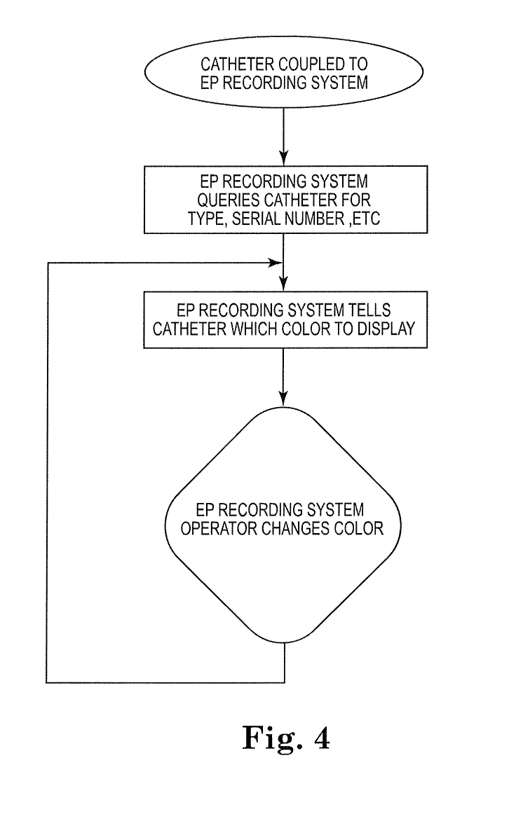

Referring now to FIG. 4 a flow chart depicting the method of identifying an interventional medical device is shown. A catheter is first coupled to an electrophysiology recording system. The electrophysiology recording system queries the catheter for type, serial number and other relevant information such as model of the catheter, configuration, optional features, calibration data, calibration constants, revision, build date, expiration date, date of last use, hours of use, permissible use duration, permissible number of uses, patient information, failure codes, single use enforcement, calibration settings, statistical information. The requested information is then transmitted from the memory structure in the microcontroller through the bidirectional cable to the electrophysiology recording system. The electrophysiology recording system then sends a signal to the microcontroller indicating which color to display for the connected catheter. The microcontroller then sends a signal to the LED to display the requested color. As additional catheters are coupled to the EP system the process is repeated.

Those of skill in the art will appreciate that the system in accordance with the invention may be in whole or in part MR compatible.

We claim:

1. A system for identifying an interventional medical device comprising:
a catheter handle including a housing having a lens thereon, a visual identification system housed within said housing, and a microcontroller with a memory structure operable to control said visual identification system.

2. The system of claim 1 wherein said visual identification system comprises a light emitting diode structured to emit a plurality of colors, a configurable textual display, a graphic display and combinations of the foregoing.

3. The system of claim 1 wherein said lens is transparent or semi-transparent.

4. The system of claim 1 wherein said catheter handle further includes a communications interface.

5. The system of claim 4 further comprising a catheter operably coupled to said catheter handle.

6. The system of claim 5 further comprising an electrophysiology recording system.

7. The system of claim 6 further comprising a bi-directional communications cable in operable communication with said communications interface and said electrophysiology recording system.

8. The system of claim 7 wherein said memory structure includes information about the catheter selected from type of catheter, configuration, calibration constants, serial number, revision, build date, expiration date, date of last use, hours of use, permissible use duration, permissible number of uses, patient information, failure codes, single use enforcement, calibration settings, statistical information and combinations of the foregoing.

9. The system of claim 8 wherein said microcontroller is operable to communicate the catheter information stored in said memory structure to the electrophysiology recording system.

10. The system of claim 9 wherein said electrophysiology recording system is operable to communicate a desired color for the catheter to the microcontroller.

11. The system of claim 10 wherein said microcontroller is operable to transmit a signal indicating a desired color to the light emitting diode and the light emitting diode displays said desired color through said lens indicating a particular catheter to a user.

12. A system for identifying an interventional medical device comprising:
a plurality of catheter handles each including a housing having a lens thereon, a visual identification system housed within said housing, and a microcontroller with a memory structure operable to control said visual identification system.

13. The system of claim 12 wherein each of said visual identification systems comprises a light emitting diode structured to emit a plurality of colors.

14. The system of claim 12 wherein each of said lenses is transparent or semi-transparent.

15. The system of claim 12 wherein each of said catheter handles further includes a communications interface.

16. The system of claim 15 further comprising a plurality of catheters each of which operably coupled to one of said catheter handles.

17. The system of claim 16 further comprising at least one electrophysiology recording system.

18. The system of claim 17 further comprising a plurality of bi-directional communications cables each in operable communication with each of said communications interfaces and said at least one electrophysiology recording system.

19. The system of claim 18 wherein said memory structure includes information about the catheter selected from type of catheter, configuration, calibration constants, serial number, revision, build date, expiration date, date of last use, hours of use, permissible use duration, permissible number of uses, patient information, failure codes, single use enforcement, calibration settings, statistical information and combinations of the foregoing.

20. The system of claim 19 wherein said memory structure is operable to communicate the catheter information of said plurality of catheters to the electrophysiology recording system.

21. The system of claim 20 wherein said electrophysiology recording system is operable to communicate a desired color for one or more of said plurality of catheters to the microcontroller associated with said catheter handle.

22. The system of claim 21 wherein said microcontroller is operable to transmit a desired color signal to the light emitting diode and the light emitting diode displays said desired color through said lens indicating a particular functional use of said catheter to a user and further wherein a desired color of one light emitting diode is different than a desired color of any other light emitting diode.

23. The system of claim 2 wherein said configurable textual display and said configurable graphic display are displayed through said lens, on said housing or on a catheter operably coupled to said catheter handle.

24. The system of claim 9 wherein said desired color correlates to a color of a trace of electrogram produced by the catheter.

25. The system of claim 9 wherein said light emitting diode is configurable to communicate a failure code to a user.

26. A method for identifying an interventional medical device comprising:
providing a catheter operably coupled to a catheter handle, the catheter including a housing having a lens thereon, a visual identification system housed within said housing, and a microcontroller with a memory structure operable to control said visual identification system;
providing an electrophysiology recording system;
coupling said catheter to said electrophysiology recording system;
sending a query from the electrophysiology recording system to said microcontroller for information about said catheter, said information selected from catheter type, serial number model, configuration, calibration constants, revision, build date, expiration date, date of last use, hours of use, permissible use duration, permissible number of uses, patient information, failure codes, single use enforcement, calibration settings, or statistical information;
transmitting from the microcontroller through a bidirectional cable to the electrophysiology recording system the information about the catheter;
transmitting a signal from the electrophysiology system to the microcontroller indicating a desired color for said coupled catheter;
sending a signal from said microcontroller to said visual identification system to display said desired color.

27. The method of claim 26 further comprising coupling a plurality of catheters to said electrophysiology recording system and repeating said method.
